(19) United States
(12) Patent Application Publication
(10) Pub. No.: US 2023/0348743 A1
(43) Pub. Date: Nov. 2, 2023

(54) ANTIMICROBIAL INKJET COMPOSITION

(71) Applicant: Kornit Digital Ltd., Rosh HaAyin (IL)

(72) Inventors: Jacob MANN, Zoran (IL); Ran ELIASH, Tel-Aviv (IL); Allon SHIMONI, Modiin-Maccabim-Reut (IL)

(73) Assignee: Kornit Digital Ltd., Rosh HaAyin (IL)

(21) Appl. No.: 17/914,771

(22) PCT Filed: Apr. 6, 2021
(86) PCT No.: PCT/IL2021/050385

§ 371 Date: Sep. 27, 2022

Related U.S. Application Data
(60) Provisional application No. 63/005,543, filed on Apr. 6, 2020.

(51) Int. Cl. C09D 11/38; D06P 5/30; D06P 1/673

(52) U.S. Cl. CPC C09D 11/38; D06P 5/30; D06P 1/673

(57) ABSTRACT

Provided herein is a process for inkjet printing an antimicrobial layer on a textile substrate, which includes antimicrobial metallic nanoparticles, as well as an article of manufacturing obtainable by the forgoing.

### RELATED APPLICATION

This application claims the benefit of priority of U.S. Provisional Patent Application No. 63/005,543 filed Apr. 6, 2020, the contents of which are incorporated herein by reference in their entirety.

### FIELD AND BACKGROUND OF THE INVENTION

The present invention, in some embodiments thereof, relates to an inkjet process, and more particularly, but not exclusively, to an inkjet process that can be used to render printable substrate into viral-free substrates.

The usage of metal nanoparticles or metal oxides nanoparticles is known for as antiviral/microbial agents on surfaces including on textiles. The deposition of these metal/oxides on textile are typically done by immersions of all the textile in the solution or dispersion and then drying at low temp in order to prevent the dis-functionality of the antiviral/antimicrobial agents due to denaturation or due sintering of the nanoparticles that will be much less efficient in case of sintered particles that will lose the their “special” properties as nanoparticles (less than 100 nm).

Thus, one of the main challenges of antiviral/antimicrobial agents for textiles is to “bind” the antiviral/antimicrobial agents to or on the fabric and keep these properties during normal use and after washing the fabric.

Another challenge is the costs of these antiviral/antimicrobial agents, some are using costly silver and the unselective of the particles are costly as well as that the processes deposed particles into both sides as well as in the depth of the fibers, this deposition in the depth and both sides of the fabric is costly and may not be useful in some cases.

Seino, S. et al. [“Antiviral Activity of Silver Nanoparticles Immobilized onto Textile Fabrics Synthesized by Radiochemical Process”, MRS Advances, 1, 1-6, 10.1557/adv.2016.43] reported antiviral activity of metallic silver nanoparticles immobilized on textile fabrics. The paper shows that Ag nanoparticles which were synthesized by radiochemical process are firmly immobilized on the surface of support textile fabrics of cotton. Small Ag particles of about 2-4 nm were observed together with relatively large particles of more than 10 nm. The Ag nanoparticles showed antiviral activity against Influenza A and Feline Calicivirus. The antiviral activity significantly depended on the concentration of the Eagle's minimal essential medium. It was implied that the surface passivation by inhibitory agent lead to the deactivation of metallic Ag nanoparticles.

### SUMMARY OF THE INVENTION

According to an aspect of some embodiments of the present invention there is provided a process of printing an antiviral layer on a textile substrate, which is effected by:
inkjet printing a binder composition on a predetermined area of the substrate ;
inkjet printing an antimicrobial ink composition that comprises antimicrobial nanoparticles (antimicrobial NPs), on the area; and curing the area, wherein the antimicrobial NPs are having an average particle size that ranges 10-100±10% nm; and the binder is characterized by a curing temperature than ranges about 100-170° C. or by ultraviolet radiation.

In some embodiments, the curing is effected for a time period that ranges about 1-12 minutes.

In some embodiments, the antimicrobial ink composition is applied on the area at a density of up to 0.2 gr/inch².

In some embodiments, the binder composition and the antimicrobial ink composition are formulated as a single composition.

In some embodiments, the binder composition is applied on the area prior to applying the antimicrobial ink composition.

In some embodiments, the antimicrobial ink composition is applied on the area prior to applying the binder composition.

In some embodiments, the binder composition the antimicrobial ink composition are applied concomitantly on the area.

In some embodiments, the antimicrobial NPs comprise metallic, ionic and/or oxide of metals selected from the group consisting of silver, copper, zinc, gold, titanium, aluminum, and any combination thereof.

In some embodiments, the antimicrobial NPs comprise povidone iodine.

In some embodiments, the antimicrobial nanoparticles include silver nanoparticles coated with mercaptoethane sulfonate.

In some embodiments, the curing is effected by belt-conveyor oven heated by force heated air, IR lamps or UV radiation.

In some embodiments, the antimicrobial nanoparticles are selected such that the antimicrobial ink confers an antiviral effect.

According to an aspect of some embodiments of the present invention there is provided an article of manufacturing, comprising a textile substrate and an antimicrobial layer attached on at least a part of the surface of the substrate, wherein the antimicrobial layer comprises antimicrobial NPs attached to the substrate by the process provided herein.

In some embodiments, the article is a wearable garment, a personal protective equipment (PPE), a blanket, a sheet, a diaper, a mat, a hood, a curtain, a rug/carpet, a towel, or a wipe.

In some embodiments, the PPE is selected from the group consisting of a face mask, a glove, a lab coat, a head cap, a footwear, and a hood.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SOME SPECIFIC EMBODIMENTS OF THE INVENTION

The present invention, in some embodiments thereof, relates to an inkjet process, and more particularly, but not exclusively, to an inkjet process that can be used to render printable substrate into viral-free substrates.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have contemplated that using inkjet for deposition of an antimicrobial metal and/or metal oxide in a predetermined pattern on a substrate (e.g., natural, synthetic or blend fabric), would provide an efficient way to deploy these antimicrobial agents particularly on personal protective equipment articles.

The term “antimicrobial”, as used herein, encompasses “antiviral” and/or “bacterial”. The term “antimicrobial agents”, as used herein, encompasses antiviral and/or bacterial agents. In some embodiments, the term “antimicrobial agents” refers to antiviral agents. In some embodiments, the term “antimicrobial agents” refers to bacterial agents.

The ink, according to some embodiments of the present invention, is composed of metal and/or metal oxide nanoparticles (less than 100 nm) up to 5%, formulated with a suitable binder. After deposition on the fabric, the printed patters undergoes drying and curing process at elevated temp (more than 80° C.).

The printing process can be a part of other printing process on a textile and will not require another step (the color inks and antimicrobial are in the same step as well as the curing process).

This is a sustainable process because it does not require any washing process to remove excess ink or active agents and reduce water consumption in the process, as well and will not produce any waste that may need special waste treatment, this process is a dry process so no need to another step of drying.

This process is also cost effective due to fact that the antimicrobial agents are purposefully printed only on predetermined parts of the substrate, thereby saving the costly agents.

### Effective Antimicrobial Agents for Inkjet Printing

As discussed hereinabove, the efficacy of the antimicrobial agents belonging to the family of metal and metal-oxide nanoparticles have been shown, however, in the context of inkjet printing, the challenge is greater for articles of clothing and personal protective equipment (PPE), which is expected to experience wear and tear, wash cycles, stretching and rubbing. The assumption that an antimicrobial metallic particle would exert antimicrobial activity when affixed to a fabric substrate is only partially founded, as more factors come into play. For example, the requirement that the surface of the particle be at least partially exposed to the environment in which viruses are expected to exist, as well as affixed to the substrate to sustain wash and rub. In other words, the particle cannot be immersed and encapsulated in the binder that affixes it to the substrate.

In the context of embodiments of the present invention, the antimicrobial metallic/metal-oxide nanoparticles that are suitable for use in inkjet printing processes are jointly referred to herein as “antimicrobial NPs”. The efficacy of the antimicrobial NPs stems from their average size, there distribution/density, and their ability to form a matrix with a binder such that their surface is at least partially exposed after the binder has cured on the substrate. The terms “antimicrobial NPs” encompasses, without limitation, metallic silver, silver ion and silver-oxide NPs, metallic zinc, zinc ion and zinc-oxide NPs, copper, metallic copper ion and copper-oxide NPs, metallic gold, gold ion and gold-oxide NPs, metallic tin, tin ion and tin-oxide NPs, metallic titanium, titanium ion and titanium-dioxide NPs, as well as other antimicrobial metals in their metallic, ionic and oxide forms. The antimicrobial NPs may be coated or pristine, having a native oxide layer, a hydroxyl layer or a hydride layer, and any other form that maintains and sustains their antimicrobial activity. It is noted that since some of the antimicrobial NPs are antimicrobial in general, namely are also effective against other pathogens such as bacteria, the term “antimicrobial NPs” also encompasses antimicrobial and/or antifungal effect.

While reducing the present invention to practice, the inventors have found that there are some limitations to the size, density and adhesion parameters, which may be critical to the efficacy antimicrobial NPs. For example, the antimicrobial NPs should be small enough to pass a typical inkjet nozzle, but not too small so as to be completely coated with the binder and not be stable as ink. The size limitation is also critical due to the heating step that is typical to inkjet printing processes, whereas too small antimicrobial NPs may fuse/sinter and lose surface area and thus efficacy. The distribution of the antimicrobial NPs is tied to their size limitation for similar reasons, whereas too dense particles would be wasteful and prone to sintering. Finally, the antimicrobial

NPs may “float” on its surface so as to form a matrix that holds the antimicrobial NPs in place yet does not encapsulate them entirely.

Thus, according to some embodiments of the present invention, the average size of the antimicrobial NPs ranges 10-100 nm.

According to some embodiments of the present invention, the binder is from the family of acrylic or polyurethane, characterized by a curing temperature that ranges 100-170° C. For example, the binder is Appretan E2100 (ARCHROMA), Appretan E6200, ENCOR 1130S (Arkema), PURLASTIC 8192 (polysistec), and any combinations thereof.

### A Process of Inkjet Printing Antimicrobial NPs on a Fabric Substrate

As discussed hereinabove, the efficacy of antimicrobial NPs, according to embodiments of the present invention, depends on the type and density of thereof on the substrate, as well as the adhesion and form of adhesion of the antimicrobial NPs to the substrate. The printing process of the antimicrobial NPs with a binder that can be applied on the substrate as a combined or separate compositions is effected directly on the substrate by inkjet machinery and software. The curing step in the printing process is critical to the efficacy of the antimicrobial NPs, as it determines the strength of adhesion and the form of adhesion (some exposed NP surface), as well as avoidance from sintering/fusing/agglomeration of the NPs into macroscopic entities.

Thus, according to an aspect of some embodiments of the present invention, there is provided a process of printing an antimicrobial layer on a textile substrate, which is effected by:
inkjet printing a binder composition on a predetermined area of the substrate;
inkjet printing an antimicrobial ink composition that includes antimicrobial NPs, as described hereinabove, on that area; and
curing the binder by heat for a time period that ranges 1-12 minutes,
wherein:
the antimicrobial NPs having an average particle size that ranges 10-100±10% nm, or 10-50±10% nm; and
the binder is characterized by a curing temperature than ranges about 80-200° C., or about 100-170° C.

In some embodiment a pretreatment composition is deposited before, after or concomitantly to the antimicrobial ink composition in order to improve the ink laydown on the substrate. The pretreatment might be an acid or a pretreatment composition for textile such as bleaching agents, bio-polishing, acids and other well-known pretreatments for textiles.

In some embodiments, the binder composition and the antimicrobial ink composition are formulated into a combined composition. In some embodiments, the binder composition is applied simultaneously with the antimicrobial ink composition. In some embodiments the binder composition is applied on the substrate before the antimicrobial ink composition, and in some embodiments the antimicrobial ink composition is applied before the binder composition.

The antimicrobial ink composition is applied on the substrate at a density of 30-800 gr/m².

In some embodiments the antimicrobial NPs include metallic silver(Ag⁰), silver ions (Ag⁺¹) and/or silver-oxide (Ag₂O). In some embodiments the antimicrobial NPs include metallic copper(Cu⁰), copper ions (Cu(I)) and/or copper-oxide (CuO/Cu(II)). In some embodiments the antimicrobial NPs include zinc, zinc ions and/or zinc-oxide (ZnO).

In some embodiments the curing step is effected by belt-conveyor oven heated by force heated air, IR lamps or ultraviolet radiation.

### Textile Having an Antimicrobial Layer Printed Thereon

According to some embodiments of an aspect of the present invention, there is provided an article of manufacturing that includes a textile substrate and a layer of an antimicrobial NPs attached thereto, which is obtainable by an inkjet printing process as provided herein.

The article can be in any form and shape for use as a wearable garment, personal protective equipment (PPE), a blanket, a sheet, a diaper, a mat, a hood, a curtain, a rug/carpet, a towel and a wipe.

PPE encompasses any form of skin protection such as, for a non-limiting example, face masks, gloves, lab coat, head cap, booties, and the like.

It is expected that during the life of a patent maturing from this application many relevant processes for inkjet printing antimicrobial NPs-containing inkjet inks will be developed and the scope of the above terms is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental and/or calculated support in the following examples.

### EXAMPLES

Reference is now made to the following examples, which together with the above descriptions illustrate some embodiments of the invention in a non-limiting fashion.

### Example 1

### Proof of Concept

A proof of concept of some embodiments of the present invention was carried out by printing an antimicrobial ink composition that included 1 wt. % of silver nanoparticles and 0.5 wt. % of an aqueous acrylic emulsion binder (50 wt. % solids) in water, on a cotton satin fabric characterized by 127 g/m², whereas the concentration of silver NPs in the ink was up to 5 wt. %, and the amount of antimicrobial ink which was printed per unit area was up to 0.2 gr/inch². The process was finalized by curing the area on which the antimicrobial NPs were printed on for about 6 min at about 160° C.

Table 1 presents the parameters of the experimental inkjet printing process, according to some embodiments of the present invention.

TABLE 1

| | |
|---|---|
| Fabric grammage (g/m²) | 127 |
| Grammage (g/in²) | 0.082 |
| resolution cross dot/inch) | 600 |
| resolution scan (dot/inch) | 800 |
| drop volume (pL) | 30 |
| ink density (g/mL) | 1.1 |
| drop weight (ng) | 33 |
| ink weight per in² (ng/inch²) | 15840000 |
| ink weight per in² (g/inch²) | 0.01584 |
| silver content in ink | 1.0 wt. % |
| silver percent of fabric | 0.19 wt. % |

### Example 2

### Silver Nanoparticles-Containing Formulations

The following is an exemplary embodiment of the present invention, providing an ink composition comprising silver nanoparticles (NPs) dispersion (50% Ag).

Table 2 presents the silver NPs containing ink composition, referred to herein as “Formulation #1”, and some printing parameters, according to some embodiments of the present invention.

TABLE 2

| Ingredients in Formulation | #1 |
|---|---|
| Humectant (solvent) | 71 |
| Acrylic binder | 1.6 |
| DI Water | 25.7 |
| BYK 348 | 0.1 |
| silver nano particle dispersion (50% Ag) | 1.6 |
| Total | 100 |
| S.T. (mN/m) | 27.3 |
| pH | 6.15 |
| density (g/mL) (r.t.) | 1.050 |
| Viscosity (cP) @28 C., @10 and 1000 1/s | 11.0-11.7 |

Ink Formulation #1 was loaded into Kornit Storm HD printing machine equipped with Dimatix PQWR 35 pl inkjet printheads, and printed in 800×800 DPI resolution on a cotton fabric (100% woven sateen cotton; 127 g/m², Oragnsateen-504 from Top Value Fabrics Inc) treated with

Kornit fixation fluid composed of acidic pretreatment (FIXA).

Thereafter, the fabric was dried for 6 minutes at 160° C. in a hot air dryer.

Under the abovementioned printing conditions, the amount of silver placed on the fabric is approximately 0.2% silver per fabric's weight.

One sample of the abovementioned printed fabric was washed in a Bosh WAN20050IL washing machine at 40° C., spun at 1000 RPM, in a 1 hour program with Persil powder detergent.

The washed samples were sent to the Institute for Food Microbiology and Consumer Goods LTD (IFMCG) to be tested according to AATCC TM 100 “Test Method for Antibacterial Finishes on Textile Materials” and ISO 18184:2019 Textiles for determination of antimicrobial activity of these textile products for incubation period of 24 hours. The results of the antimicrobial activity are presented in Table 3 below, wherein R represents the percent reduction of viable cells according to AATCC TM 100, and Mᵥ represents the antimicrobial activity value according to ISO 18184.

TABLE 3

| | Staph. Aureus | | Klebsiella pneumonia | |
|---|---|---|---|---|
| Sample description | R | Mᵥ | R | Mᵥ |
| Untreated cotton fabric | 48.76% | 0.29 | −12.13% | −0.05 |
| Untreated cotton fabric, washed | 99.24% | 2.25 | 5.31% | 0.02 |
| #1 printed on cotton | 99.91% | 3.20 | 99.99% | 4.03 |
| #1 printed on cotton, washed | 99.97% | 3.55 | 99.99% | 4.20 |

As can be seen in Table 3, the antimicrobial activity the untreated samples was small or null, while the treated samples exhibited notable antimicrobial activity before and after washing, demonstrating that the ink formulation is capable of providing these traits to the fabric even after the fabric has been subjected to washing.

Another ink formulation containing silver nanoparticles bound by a polyurethane (PU) binder was printed and tested for antimicrobial activity. Table 4 presents the PU-based silver NPs containing ink composition, referred to herein as “Formulation #2”, and some printing parameters, according to some embodiments of the present invention.

TABLE 4

| Ingredients in Formulation | #2 |
|---|---|
| DI Water | 23.9 |
| Polyurethane binder (50%) | 1.5 |
| Blocked isocyanate | 0.2 |
| BYK 348 | 0.1 |
| Silver nano particles dispersion (10% Ag) | 4.3 |
| Humectant (solvent) | 70 |
| Total | 100 |
| S.T. (mN/m) | 25.2 |
| pH | 7.59 |
| density (g/mL) (r.t.) | 1.048 |
| Viscosity (cP) at 28° C., at 10 and 1000 1/s | 10.2-10.8 |

Formulation #2 was loaded on Kornit Storm HD printer equipped with Dimatix PQWR 35 pl print heeds and printed in 800×800 DPI resolution on the same cotton fabric as in 0 alongside Kornit fixation on the fly fluid—inkjet acidic inline pretreatment by print inkjet (FOF). This formulation in this configuration confers approximately 0.1 wt % of silver per fabric weight. The antimicrobial results are summarized in Table 5.

TABLE 5

| | Staph. Aureus | | Klebsiella pneumonia | |
|---|---|---|---|---|
| Sample description | R | Mᵥ | R | Mᵥ |
| Untreated cotton fabric, washed | 99.40% | 2.2 | 99.40% | 2.2 |
| #2 printed with FOF and washed | 99.99% | 4.1 | >99.99% | 4.2 |

A spray formulation containing silver nanoparticles coated with mercaptoethane sulfonate (Ag-MES), was tested under AATCC 100 for antimicrobial activity.

Table 6 presents the ink composition containing Ag-MES, referred to herein as “Formulation #3”, according to some embodiments of the present invention.

TABLE 6

| | Formula number |
|---|---|
| Ingredient | 3# |
| DI Water | 98.65 |
| Polyurethane binder (50%) | 0.8 |
| Blocked isocyanate | 0.1 |
| BYK 348 | 0.05 |
| Ag-MES | 0.4 |
| Total | 100 |

Formulation #3 was sprayed on cotton fabric (same as in previous examples) using a spray gun to give 0.45 g/100 cm² of wet formulation. In addition, Formulation #3 was sprayed on a cotton that FOF was printed on in 800×800 DPI. The fabrics were dried for 6 minutes at 160° in a hot air dryer, and the results are presented in Tale 7 below.

TABLE 7

| | Staph. Aureus | | Klebsiella pneumonia | |
|---|---|---|---|---|
| Sample description | R | Mᵥ | R | Mᵥ |
| cotton (reference) | 93.00% | 1.2 | 35% | 0.2 |
| cotton (washed) | 72.35% | 0.6 | 95.50% | 1.3 |
| #3 + FOF on cotton | >99.9% | 3.9 | >99% | 2.3 |
| #3 + FOF on cotton, washed | >99.9% | 3.5 | >99.9% | 3.3 |
| #3 on cotton | >99.9% | 3.8 | >99.9 | 3.6 |
| #3 on cotton, Washed | >99.9% | 3.9 | >99 | 2.9 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

In addition, any priority document(s) of this application is/are hereby incorporated herein by reference in its/their entirety.

1. A process of printing an antimicrobial layer on a textile substrate, comprising:
inkjet printing a binder composition on a predetermined area of the substrate;
inkjet printing an antimicrobial ink composition that comprises antimicrobial nanoparticles, on said area; and
curing said area,
wherein:
the antimicrobial nanoparticles are having an average particle size that ranges 10-100±10% nm; and
said binder is characterized by a curing temperature than ranges about 100-170° C. or by ultraviolet radiation.

2. The process of claim 1, wherein said curing is effected for a time period that ranges about 1-12 minutes.

3. The process of claim 1, wherein said antimicrobial ink composition is applied on said area at a density of up to 0.2 gr/inch².

4. The process of claim 1, wherein said binder composition and said antimicrobial ink composition are formulated as a single composition.

5. The process of claim 1, wherein said binder composition is applied on said area prior to applying said antimicrobial ink composition.

6. The process of claim 1, wherein said antimicrobial ink composition is applied on said area prior to applying said binder composition.

7. The process of claim 1, wherein said binder composition said antimicrobial ink composition are applied concomitantly on said area.

8. The process of claim 1, wherein said antimicrobial nanoparticles comprise metallic, coated metallic, ionic and/or oxide of metals selected from the group consisting of silver, copper, zinc, gold, titanium, aluminum, and any combination thereof.

9. The process of claim 1, wherein said antimicrobial nanoparticles comprise povidone iodine.

10. The process of claim 1, wherein said antimicrobial nanoparticles comprise silver nanoparticles coated with mercaptoethane sulfonate.

11. The process of claim 1, wherein curing is effected by belt-conveyor oven heated by force heated air, IR lamps or UV radiation.

12. The process of claim 1, wherein said antimicrobial nanoparticles are selected such that said antimicrobial ink confers an antiviral effect.

13. An article of manufacturing, comprising a textile substrate and an antimicrobial layer attached on at least a part of the surface of the substrate, wherein said antimicrobial layer comprises antimicrobial nanoparticles attached to the substrate by the process of claim 1.

14. The article of claim 13, selected from the group consisting of a wearable garment, a personal protective equipment (PPE), a blanket, a sheet, a diaper, a mat, a hood, a curtain, a rug/carpet, a towel, and a wipe.

15. The article of claim 14, wherein said PPE is selected from the group consisting of a face mask, a glove, a lab coat, a head cap, a footwear, and a hood.
